(19) United States
(12) Patent Application Publication
(10) Pub. No.: US 2025/0376655 A1
(43) Pub. Date: Dec. 11, 2025

(54) Distribution Measurement Device

(71) Applicant: Terumo Kabushiki Kaisha, Tokyo (JP)

(72) Inventors: Itaru OKUBO, Kanagawa (JP); Liming LIU, Kanagawa (JP)

(21) Appl. No.: 19/310,074

(22) Filed: Aug. 26, 2025

Related U.S. Application Data
(63) Continuation of application No. PCT/JP2024/007194, filed on Feb. 28, 2024.

(30) Foreign Application Priority Data
Feb. 28, 2023 (JP) 2023-029014
Feb. 28, 2023 (JP) 2023-029016

(51) Int. Cl. C12M 1/34; C12M 1/32; C12M 1/36

(52) U.S. Cl. CPC C12M 41/36; C12M 23/12; C12M 41/48

(57) ABSTRACT

A distribution measurement device includes: a plurality of electrodes provided in a bioreactor, a selection unit that is configured to selects a pair of the electrodes from the plurality of electrodes, and a measurement unit that is configured to measure a physical quantity between the pair of electrodes selected by the selection unit.

### CROSS-REFERENCE TO RELATED APPLICATIONS

The present application is a continuation of the International Patent Application No. PCT/JP2024/007194 filed Feb. 28, 2024, which designated the U.S. and claims the benefit of priority from Japanese Patent Application Nos. JP20230029014 and JP20230029016 filed Feb. 28, 2023. The entire disclosures of the above-identified applications are incorporated herein by reference.

### FIELD

The present disclosure relates to a distribution measurement device that measures a distribution of cells and similar materials within a bioreactor.

### BACKGROUND

JP6381083B discloses a technique for measuring a cell density in a culture medium during cell culture.

To optimize cell culture, a technique for grasping a diffusion state of cells inside a bioreactor is desired.

### SUMMARY

At least one example embodiment relates to distribution measurement device. The distribution measure device may include a plurality of electrodes in a bioreactor, a selection unit that selects a pair of the electrodes from the plurality of electrodes, and a measurement unit that measures a physical quantity between the pair of electrodes.

In at least one example embodiment, the plurality of electrodes may be disposed along a longitudinal direction of the bioreactor.

In at least one example embodiment, two or more pairs of electrodes define a plurality of cell concentration measurement ranges along the longitudinal direction in the bioreactor such that the distribution of cells in the longitudinal direction of the bioreactor can be measured. The distribution of cells in the longitudinal direction of the bioreactor contributes to optimization of cell culture.

In at least one example embodiment), an electrode group including the plurality of electrodes may include a plurality of assembled electrodes, and the plurality of assembled electrodes may be disposed apart from each other along the longitudinal direction of the bioreactor.

In at least one example embodiment, a longitudinal direction of each of the electrodes of the plurality of electrodes may intersect the longitudinal direction of the bioreactor.

In at least one example embodiment, the physical quantity may be electrostatic capacitance.

In at least one example embodiment, a hollow fiber may be provided in the bioreactor, and a longitudinal direction of the hollow fiber may be along the longitudinal direction of the bioreactor.

In at least one example embodiment, the bioreactor may be provided with a plurality of supply and discharge ports. For example, a first supply and discharge port of the plurality of supply and discharge ports may be provided at one end of the bioreactor, and a second supply and discharge port of the plurality of supply and discharge ports may be provided at the other end of the bioreactor.

In at least one example embodiment, the distribution measurement device may further include a display control unit that displays (for example, on a display unit) information indicating a distribution of cells in the longitudinal direction of the bioreactor based on the physical quantity measured by the measurement unit.

In at least one example embodiment, the distribution measurement device may further include a storage control unit that stores (for example, in a storage unit) information indicating a distribution of cells in the longitudinal direction of the bioreactor based on the physical quantity measured by the measurement unit.

In at least one example embodiment the distribution measurement device may further include a control unit that controls at least one of an inflow rate of a culture medium into the bioreactor and an outflow rate of the culture medium from the bioreactor such that a distribution of cells in the longitudinal direction of the bioreactor becomes uniform.

In at least one example embodiment, the control unit may execute controls such that the distribution of the cells in the longitudinal direction of the bioreactor become uniform. When the distribution of the cells in the longitudinal direction of the bioreactor is uniform, nutrition may be uniformly distributed to the cells inside the bioreactor, allowing for optimization of the cell culture.

In at least one example embodiment, a longitudinal direction of the electrode may be along a longitudinal direction of the bioreactor.

In at least one example embodiment, the longitudinal direction of the electrode for measuring the physical quantity may be along the longitudinal direction of the bioreactor and by sequentially switching a combination of the electrodes, the distribution of cells in the direction intersecting the longitudinal direction of the bioreactor can be measured. The distribution of cells in the direction intersecting the longitudinal direction of the bioreactor helps to optimize the cell culture.

In at least one example embodiment, the physical quantity may be electrostatic capacitance.

In at least one example embodiment, a hollow fiber may be provided in the bioreactor, and a longitudinal direction of the hollow fiber may be along the longitudinal direction of the bioreactor.

In at least one example embodiment, the distribution measurement device may further include a display control unit that displays (for example, on a display unit) information indicating a distribution of cells in a direction intersecting the longitudinal direction of the bioreactor based on the physical quantity sequentially measured by the measurement unit.

In at least one example embodiment, the distribution measurement device may further include a storage control unit that stores (for example, in a storage unit) information indicating distribution of cells in a direction intersecting the longitudinal direction of the bioreactor based on the physical quantity sequentially measured by the measurement unit.

In at least one example embodiment, the distribution of cells in the longitudinal direction of the bioreactor or the distribution of cells in the direction intersecting the longitudinal direction of the bioreactor can be measured.

### DRAWINGS

FIG. 1 is a block diagram of an example distribution measurement device as in communication with a bioreactor in accordance with at least one example embodiment.

FIGS. 2A and 2B are schematic diagrams illustrating an example arrangement of a plurality of electrodes in the bioreactor of FIG. 1 in accordance with at least one example embodiment.

FIGS. 3A and 3B are schematic diagrams illustrating another example arrangement of the plurality of electrodes in the bioreactor of FIG. 1 in accordance with at least one example embodiment.

FIGS. 4A and 4B are schematic diagrams illustrating an example arrangement of an assembled electrode in the bioreactor of FIG. 1 in accordance with at least one example embodiment.

FIG. 5 is a schematic diagram illustrating another example arrangement of an assembled electrode in the bioreactor of FIG. 1 in accordance with at least one example embodiment.

FIGS. 6A and 6B are schematic diagrams illustrating other example arrangements of the plurality of electrodes in the bioreactor of FIG. 1 in accordance with at least one example embodiment.

FIGS. 7A and 7B are schematic diagrams illustrating other example arrangements of the plurality of electrodes in the bioreactor of FIG. 1 in accordance with at least one example embodiment.

FIGS. 8A and 8B are schematic diagrams illustrating other example arrangements of the plurality of electrodes in the bioreactor of FIG. 1 in accordance with at least one example embodiment.

FIG. 9 is a schematic diagram illustrating another example arrangement of the plurality of electrodes in the bioreactor of FIG. 1 in accordance with at least one example embodiment.

FIGS. 10A and 10B are schematic diagrams illustrating example patterns of combinations of four electrodes as illustrated in FIG. 9.

FIG. 11 is a flowchart illustrating an operation of the distribution measurement device of FIG. 1 in accordance with at least one example embodiment.

FIG. 12 is a fluid circuit diagram of another example distribution measurement device as in communication with a bioreactor in accordance with at least one example embodiment.

FIG. 13 is a block diagram of the distribution measurement device of FIG. 12.

FIG. 14 is a flowchart illustrating an operation of the distribution measurement device of FIG. 12 in accordance with at least one example embodiment.

FIG. 15 is a block diagram of another example distribution measurement device as in communication with a bioreactor in accordance with at least one example embodiment.

FIGS. 16A and 16B are schematic diagrams illustrating arrangements of a plurality of electrodes in the bioreactor of FIG. 15 in accordance with at least one example embodiment.

FIG. 17 is a flowchart illustrating an operation of the distribution measurement device of FIG. 15 in accordance with at least one example embodiment.

FIG. 18 is a schematic diagram illustrating another example arrangement of the plurality of electrodes in the bioreactor of FIG. 15 in accordance with at least one example embodiment.

FIGS. 19A and 19B are schematic diagrams illustrating another example arrangements of the plurality of electrodes in the bioreactor of FIG. 15 in accordance with at least one example embodiment.

### DETAILED DESCRIPTION

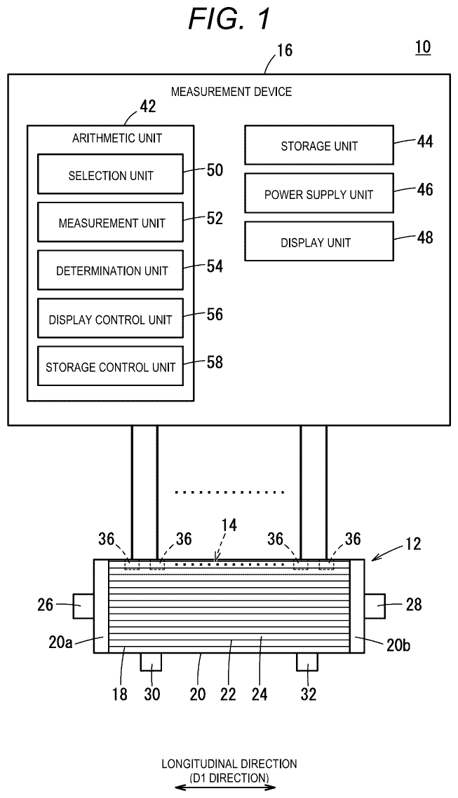

FIG. 1 is a block diagram of an example distribution measurement device 10 in accordance with at least one example embodiment. The distribution measurement device 10 as illustrated in FIG. 1 may include the bioreactor 12, an electrode group 14, and a measurement device 16. The distribution measurement device 10 as illustrated in FIG. 1 may be configured to measure a distribution of cells (e.g., embryonic stem (ES) cells, induced pluripotent stem (iPS) cells, mesenchymal stem cells, or any combination thereof) in a longitudinal direction of a bioreactor 12. Although embryonic stem (ES) cells, induced pluripotent stem (iPS) cells, and mesenchymal stem cells are discussed herein, it should be appreciated, that in various other example embodiment, the distribution measurement device 10 as illustrated in FIG. 1 may be used to measure a distribution of other cells, including, for example, yeast.

The bioreactor 12 may be a component of a cell culture device (not illustrated). The bioreactor 12 may include a plurality of hollow fiber membranes 18 and a housing 20 that supports and houses the plurality of hollow fiber membranes 18. The housing 20 may have a cylindrical shape. A longitudinal direction of each of the hollow fiber membranes 18 may be along the longitudinal direction of the bioreactor 12. That is, the hollow fiber membrane 18 may extend along the longitudinal direction of the bioreactor 12. For example, a first end portion of the hollow fiber membrane 18 may be fixed to a first end portion 20a of the housing 20 in the longitudinal direction, and a second end portion of the hollow fiber membrane 18 may be fixed to a second end portion 20b of the housing 20 in the longitudinal direction. The hollow fiber membrane 18 may be formed using polymeric materials. The hollow fiber membrane 18 may include a plurality of pores (not illustrated).

The bioreactor 12 may include a first region 22 and a second region 24. The first region 22 may include a space inside the hollow fiber membrane 18, while the second region 24 may include a space between an outer peripheral surface of the hollow fiber membrane 18 and an inner peripheral surface of the housing 20. The first region 22 and the second region 24 may communicate with each other via the plurality of pores of the hollow fiber membranes 18.

The housing 20 may include a first port 26 (e.g., first supply and discharge port), a second port 28 (e.g., second supply and discharge port), a third port 30, and a fourth port 32. The first port 26 may be disposed at the first end portion 20a of the housing 20. The first port 26 may be connected to the first end portion of each of the hollow fiber membranes 18. Accordingly, the first port 26 may communicate with the first region 22. The second port 28 may be disposed at the second end portion 20b of the housing 20. The second port 28 may be connected to the second end portion of each of the hollow fiber membranes 18.

Accordingly, the second port 28 may communicate with the first region 22.

The third port 30 and the fourth port 32 may be disposed on an outer peripheral surface of the housing 20. The third port 30 may be disposed between the first port 26 and a central portion of the housing 20 in the longitudinal direction. The fourth port 32 may be disposed between the second port 28 and the central portion of the housing 20 in the longitudinal direction, the third port 30 and the fourth port 32 may both communicate with the second region 24.

A cell suspension containing cells may be supplied from the first port 26 or the second port 28 to an inside of the bioreactor 12, and more specifically, to the first region 22. A culture medium may be supplied from the first port 26 or the second port 28 to the inside of the bioreactor 12, and more specifically, to the first region 22. The culture medium may be supplied from the third port 30 or the fourth port 32 to the inside of the bioreactor 12, and more specifically, to the second region 24. The culture medium supplied to the bioreactor 12 may move between the first region 22 and the second region 24 via the plurality of pores of the hollow fiber membrane 18.

The electrode group 14 may include a plurality of electrodes 36. The plurality of electrodes 36 may include three or more electrodes 36. The plurality of electrodes 36 may be disposed inside the bioreactor 12 along the longitudinal direction of the bioreactor 12.

The measurement device 16 may measure electrostatic capacitance and/or dielectric constant between a pair of electrodes 36. The electrostatic capacitance between the pair of electrodes 36 is a physical quantity proportional to a cell concentration (i.e., the number of cells) between the pair of electrodes 36. In at least one example embodiment, the measurement device 16 may bean impedance analyzer or an LCR meter. The measurement device 16 may include, for example, an arithmetic unit 42, a storage unit 44, a power supply unit 46, and a display unit 48.

In at least one example embodiment, the arithmetic unit 42 may be implemented by a processor such as a central processing unit (CPU) or a graphics processing unit (GPU). That is, the arithmetic unit 42 may be implemented by a processing circuit.

The arithmetic unit 42 may include a selection unit 50, a measurement unit 52, a determination unit 54, a display control unit 56, and a storage control unit 58. Each of the selection unit 50, the measurement unit 52, the determination unit 54, the display control unit 56, and the storage control unit 58 may be implemented by the arithmetic unit 42 executing a program stored in the storage unit 44.

In at least one example embodiment, at least a part of the selection unit 50, the measurement unit 52, the determination unit 54, the display control unit 56, and the storage control unit 58 may be implemented by an integrated circuit such as an application specific integrated circuit (ASIC) or a field-programmable gate array (FPGA). In at least one example embodiment, at least a part of the selection unit 50, the measurement unit 52, the determination unit 54, the display control unit 56, and the storage control unit 58 may be implemented by an electronic circuit including a discrete device.

The selection unit 50 may select a pair of electrodes 36 from the plurality of electrodes 36. In at least one example embodiment, the selection unit 50 may sequentially select a pair of electrodes 36 from the plurality of electrodes 36. The measurement unit 52 may execute switching control of the electrode 36 based on a selection result of the selection unit 50. The measurement unit 52 may measure electrostatic capacitance between the pair of electrodes 36 selected by the selection unit 50. In at least one example embodiment, the measurement unit 52 may sequentially measure electrostatic capacitance between the pair of electrodes 36 selected by the selection unit 50. The determination unit 54 may perform various determinations. The display control unit 56 may execute display control. For example, the display control unit 56 displays (for example, on the display unit 48) information indicating a distribution of cells in the longitudinal direction of the bioreactor 12 based on the electrostatic capacitance measured by the measurement unit 52. The storage control unit 58 may execute storage control. For example, the storage control unit 58 stores (for example, in the storage unit 44) information indicating the distribution of cells in the longitudinal direction of the bioreactor 12 based on the electrostatic capacitance measured by the measurement unit 52.

The storage unit 44 may include a volatile memory (not illustrated) and a non-volatile memory (not illustrated). Examples of the volatile memory include a random access memory (RAM). The volatile memory may be used as a working memory of the processor and may temporarily store data and the like necessary for processing or calculation. Examples of the non-volatile memory include a read only memory (ROM) and a flash memory. The non-volatile memory may be used as a memory for storage and may store programs, tables, maps, and the like. In at least one example embodiment, at least a part of the storage unit 44 may be in the processor, the integrated circuit, or the like as described above.

The storage unit 44 may store information indicating the distribution of cells in the longitudinal direction of the bioreactor 12 according to the storage control executed by the storage control unit 58 of the arithmetic unit 42. In at least one example embodiment, a part of the storage unit 44 may be provided outside the measurement device 16.

The power supply unit 46 may include a power supply circuit capable of applying a voltage (or current) between the pair of electrodes 36. The power supply circuit may include a plurality of switches. Each of the switches may be configured to switch connection and disconnection between a power supply (not illustrated) and the electrode 36 according to a switching signal output from the arithmetic unit 42 (measurement unit 52).

The display unit 48 may include a drive circuit and a display. The display unit 48 may display information indicating the distribution of cells in the longitudinal direction of the bioreactor 12 according to the display control executed by the arithmetic unit 42 (display control unit 56). For example, the display unit 48 may display the cell concentration in a numerical value. Alternatively, the display unit 48 may display the cell concentration as a graph.

The longitudinal direction of the bioreactor 12 is referred to as a D1 direction. Among radial directions of the bioreactor 12, two directions that are perpendicular to each other are referred to as a D2 direction and a D3 direction. The D2 direction and the D3 direction are perpendicular to the D1 direction.

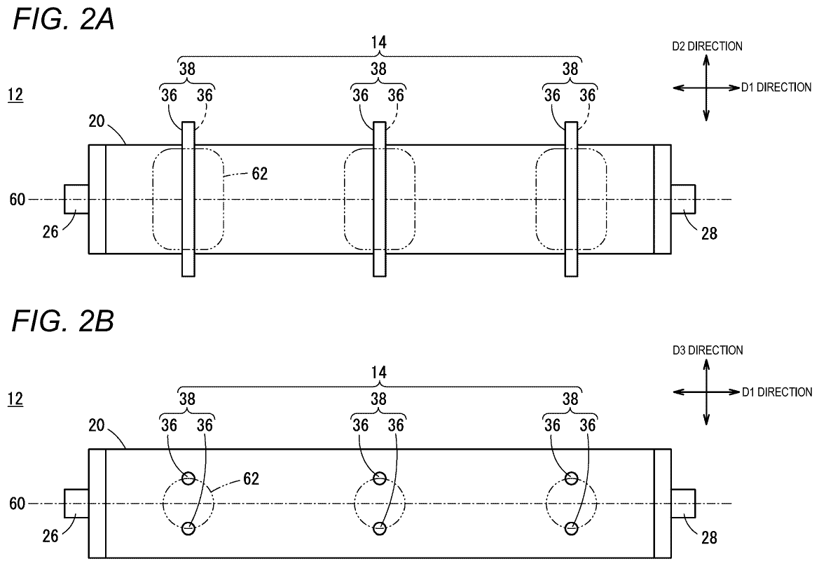

FIGS. 2A and 2B are schematic diagrams illustrating an example arrangement of the plurality of electrodes 36 in the bioreactor 12 as illustrated in FIG. 1. FIG. 2A illustrates a position relationship between the bioreactor 12 and the plurality of electrodes 36 in a plan view of the bioreactor 12. FIG. 2B illustrates a position relationship between the bioreactor 12 and the plurality of electrodes 36 in a front view of the bioreactor 12.

As illustrated in FIG. 2A, each of the electrodes 36 may extend along the D2 direction. That is, the electrode 36 may extend along a direction intersecting the D1 direction. The electrode 36 may penetrate the housing 20 of the bioreactor 12. The electrode 36 may be insulated with respect to the housing 20. The electrode 36 may be connected to the power supply unit 46 as illustrated in FIG. 1.

As illustrated in FIG. 2B, two electrodes 36 of the plurality of electrodes 36 may be disposed along the D3 direction. These two electrodes 36 may be spaced apart from each other to sandwich an axis 60 of the housing 20. When the electrostatic capacitance is measured, these two electrodes 36 may be simultaneously selected. One of the two electrodes 36 may be a positive electrode, and the other may be a negative electrode. That is, when the electrostatic capacitance is measured, these two electrodes 36 form a pair. The two electrodes 36 that form a pair and where one electrode 36 is correlated with one electrode 36 in advance is referred to as an “assembled electrode 38”. The electrode group 14 may include a plurality of assembled electrodes 38. The plurality of assembled electrodes 38 may be spaced apart from each other along the D1 direction. For example, the assembled electrodes 38 may be disposed at equal intervals along the D1 direction.

When the power supply unit 46 as illustrated in FIG. 1 applies a voltage (or a current) to the assembled electrode 38 (the pair of electrodes 36), the assembled electrode 38 stores a charge corresponding to a cell concentration between the electrodes 36. Note that the cell concentration may be substantially constant in a certain range centered on the assembled electrode 38. The assembled electrode 38 functions as a sensor member for measuring the cell concentration in a certain range. This certain range is referred to as a measurement range 62. The measurement range 62 is determined by a distance between the positive electrode and the negative electrode of the assembled electrode 38 (inter-electrode distance) and a length of each electrode 36. The length of the electrode 36 and the inter-electrode distance are preferably set such that the measurement range 62 is wide. From a viewpoint of reducing the number of electrodes 36, the length of the electrode 36 and the inter-electrode distance are preferably set such that the measurement range 62 is maximized. Further, the plurality of assembled electrodes 38 are preferably disposed along the D1 direction such that two measurement ranges 62 adjacent to each other are as close as possible. When these conditions are satisfied, the distribution of the cell concentration in the D1 direction in the bioreactor 12 can be measured with the minimum number of assembled electrodes 38. These conditions are not essential.

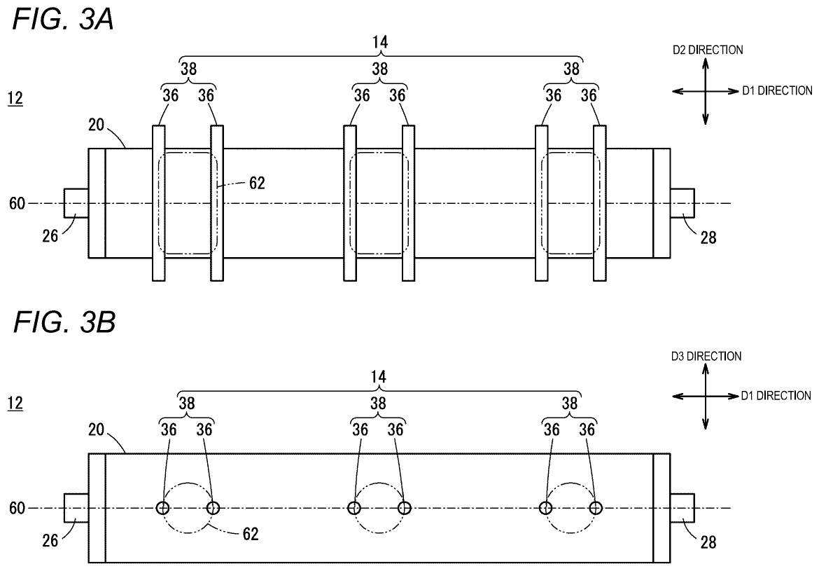

FIGS. 3A and 3B are schematic diagrams illustrating an example arrangement of the plurality of electrodes 36 in the bioreactor 12 as illustrated in FIG. 1. FIG. 3A illustrates a position relationship between the bioreactor 12 and the plurality of electrodes 36 in the plan view of the bioreactor 12. FIG. 3B illustrates a position relationship between the bioreactor 12 and the plurality of electrodes 36 in the front view of the bioreactor 12. The arrangement of the electrodes 36 as illustrated in FIGS. 3A and 3B is different from the arrangement of the electrodes 36 as illustrated in FIGS. 2A and 2B in a disposition direction of the two electrodes 36 in one assembled electrode 38.

As illustrated in FIG. 3B, the plurality of electrodes 36 may be disposed along the D1 direction. Preferably, all the electrodes 36 may intersect the axis 60 of the housing 20. For example, all the electrodes 36 may be perpendicular to the axis 60 of the housing 20. All electrodes 36 or some of the electrodes 36 may deviate from the axis 60. The electrode 36 forms an assembled electrode 38 (positive electrode and negative electrode) with the most adjacent electrode 36. In this way, the electrode group 14 includes a plurality of assembled electrodes 38. The plurality of assembled electrodes 38 may be spaced apart from each other along the D1 direction. For example, the assembled electrodes 38 may be disposed at equal intervals along the D1 direction.

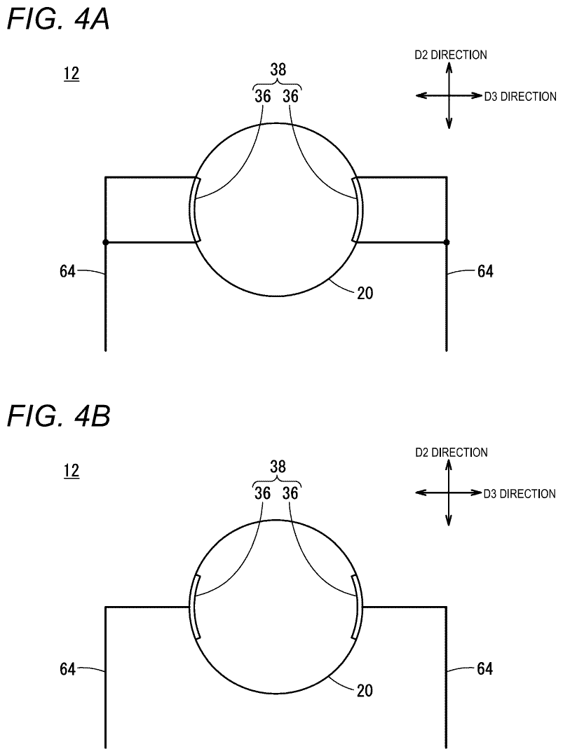

FIGS. 4A and 4B are schematic diagrams illustrating another example arrangement of the assembled electrode 38 in the bioreactor 12 as illustrated in FIG. 1. FIGS. 4A and 4B illustrate a position relationship between the bioreactor 12 and one assembled electrode 38 in a side view of the bioreactor 12. The third arrangement example is a modification of the first arrangement example. The arrangement of the assembled electrode 38 as illustrated in FIGS. 4A and 4B is different from the assembled electrode 28 as illustrated in FIGS. 2A and 2B in a shape of the two electrodes 36 forming one assembled electrode 38.

As illustrated in FIG. 4A, the electrode 36 may be curved along the inner peripheral surface of the housing 20 of the bioreactor 12. The electrode 36 may be attached to the inner peripheral surface of the housing 20. The electrode 36 may be insulated with respect to the housing 20. The electrode 36 may be connected to the power supply unit 46 as illustrated in FIG. 1) via a lead wire 64. As illustrated in FIG. 4A, the lead wire 64 may be drawn out from both end portions of the electrode 36. As illustrated in FIG. 4B, the lead wire 64 may be drawn out from a part of the electrode 36.

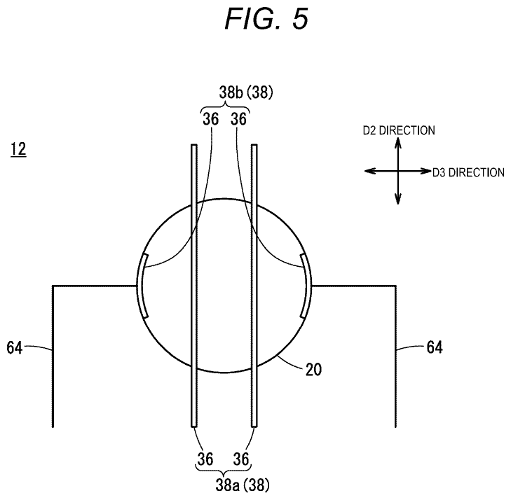

FIG. 5 is a schematic diagram illustrating another example arrangement of one assembled electrode 38 in the bioreactor as illustrated in FIG. 1. The arrangement of the assembled electrode 28 in FIG. 5 is different from the arrangement of the assembled electrode 38 as illustrated in FIGS. 2A and 2B (hereinafter referred to as a first assembled electrode 38a) and the arrangement of the assembled electrode 38 as illustrated in FIGS. 3A and 3B (hereinafter referred to as a third assembled electrode 38b) in that the first assembled electrode 38a is disposed between the electrodes of the third assembled electrode 38b.

In the instance of FIG. 5, the power supply unit 46 may apply a voltage (or current) to the third assembled electrode 38b and may measure the electrostatic capacitance of the first assembled electrode 38a and an influence of an electric double layer may be reduced.

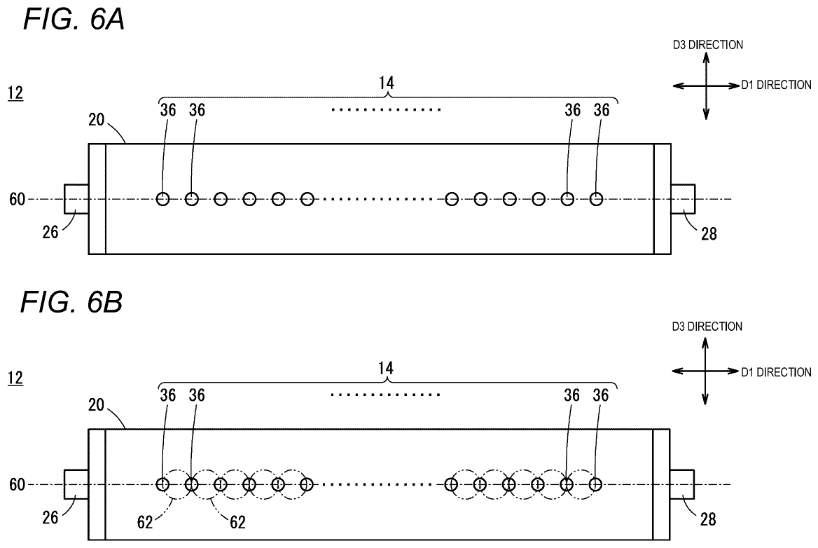

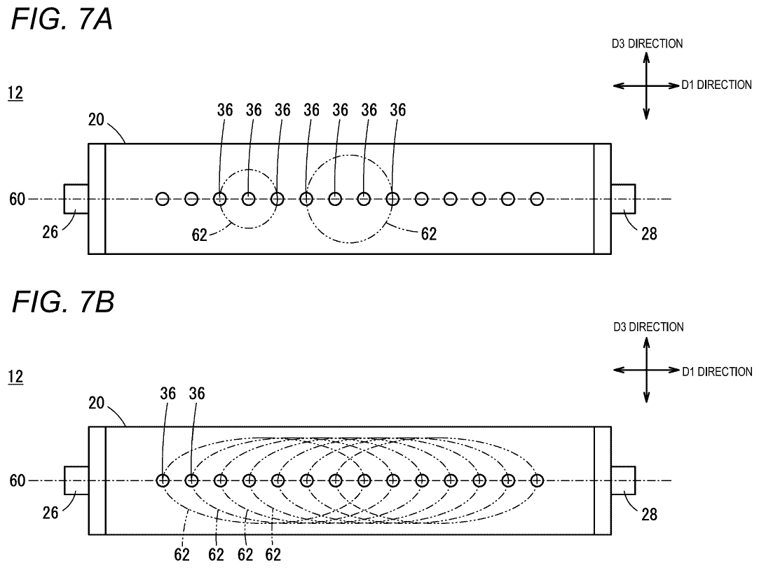

FIGS. 6A, 6B, 7A, and 7B are schematic diagrams illustrating arrangements of the plurality of electrodes 36 in the instance of FIG. 5. As illustrated in FIG. 6A, the plurality of electrodes 36 may be disposed at equal intervals along the D1 direction. In the instance of FIG. 5, the selection unit 50 as illustrated in FIG. 1 can select any two electrodes 36.

As illustrated in FIG. 6B, the selection unit 50 may sequentially select two electrodes 36 adjacent to each other as a pair of electrodes 36. As illustrated in FIG. 7A, the selection unit 50 may sequentially select two electrodes 36 sandwiching one or more electrodes 36 as a pair of electrodes 36. As illustrated in FIG. 7B, the selection unit 50 may sequentially select the pair of electrodes 36 such that a plurality of measurement ranges 62 overlap each other. In the instance of FIG. 5, the measurement range 62 can be expanded in the D1 direction, the D2 direction, and the D3 direction.

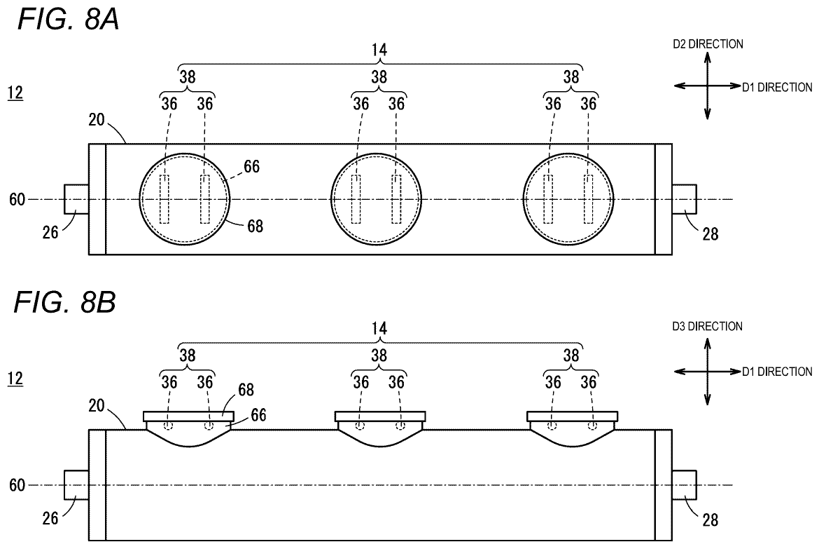

FIGS. 8A and 8B are schematic diagrams illustrating another example arrangements of the plurality of electrodes 36. FIG. 8A illustrates a position relationship between the bioreactor 12 and the plurality of electrodes 36 in the plan view of the bioreactor 12. FIG. 8B illustrates a position relationship between the bioreactor 12 and the plurality of electrodes 36 in the front view of the bioreactor 12.

As illustrated in FIG. 8B, the housing 20 of the bioreactor 12 may include a plurality of convex portions 66. Each of the plurality of convex portions 66 may protrude in the same direction (D3 direction) from the outer peripheral surface (for example, an upper surface) of the housing 20. The plurality of convex portions 66 may be spaced apart from each other along the D1 direction. Each of the convex portions 66 may be hollow. A lid portion 68 may be attachable to and detachable from a tip of the convex portion 66. The lid portion 68 may be parallel to the D2 direction and perpendicular to the D3 direction when attached to the convex portion 66. The lid portion 68 may have two electrodes 36 disposed thereon. The two electrodes 36 may be parallel to each other. For example, each of the electrodes 36 may extend along the D2 direction. The two electrodes 36 may be disposed on the lid portion 68 form an assembled electrode 38.

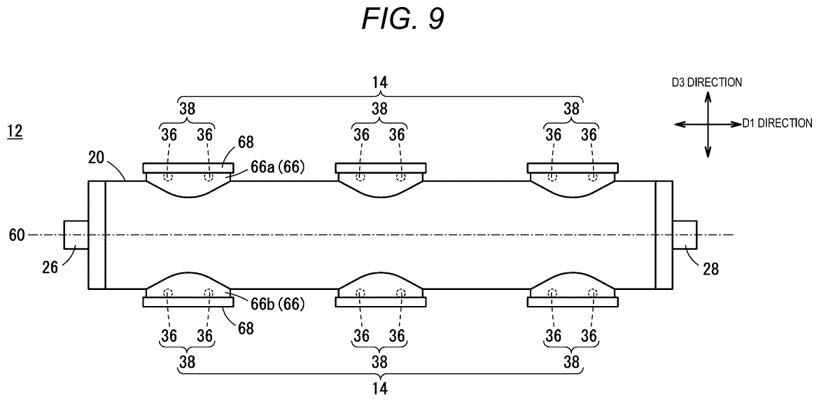

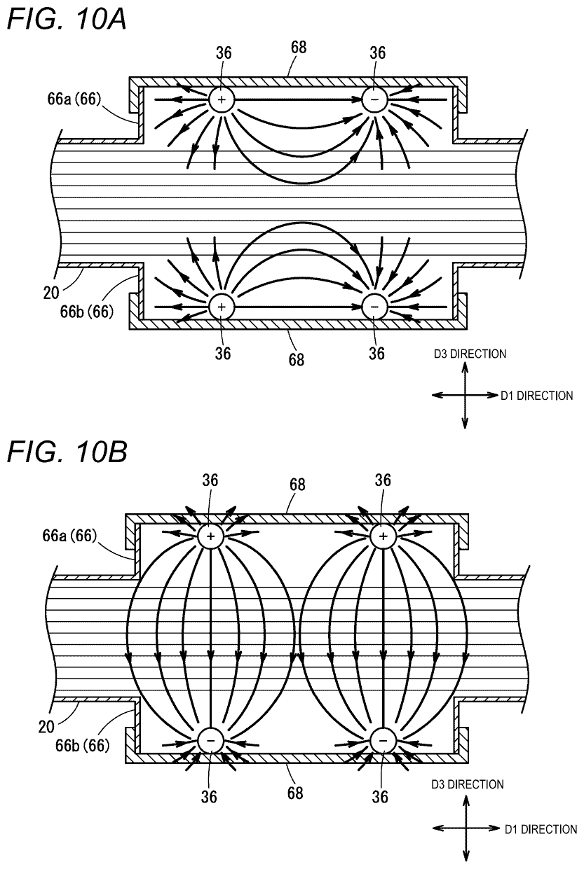

FIGS. 9, 10A, and 10B is a schematic diagram illustrating another arrangement 41 the plurality of electrodes 36. FIG. 9 illustrates a position relationship between the bioreactor 12 and the plurality of electrodes 36 in the front view of the bioreactor 12. FIGS. 10A and 10B are schematic diagrams illustrating patterns of combinations of four electrodes 36.

Similar to the bioreactor 20 illustrated in FIG. 8, the housing 20 of the bioreactor 12 as illustrated in FIG. 9 may include a plurality of convex portions 66.

As illustrated in FIG. 9, the plurality of convex portions 66 may be divided into two groups. Each of the convex portions 66 in a first group (hereinafter referred to as a first convex portion 66a) may protrude from the upper surface of the housing 20 along the D3 direction.

Each of the convex portions 66 in a second group (hereinafter referred to as a second convex portion 66b) may protrude from a lower surface of the housing 20 along the D3 direction. One first convex portion 66a and one second convex portion 66b may be disposed along the D3 direction. Further, one of the two electrodes 36 in the first convex portion 66a and one of the two electrodes 36 in the second convex portion 66b may be disposed along the D3 direction. Similarly, the other of the two electrodes 36 in the first convex portion 66a and the other of the two electrodes 36 in the second convex portion 66b may be disposed along the D3 direction.

As illustrated in FIG. 10A, the selection unit 50 may select the two electrodes 36 disposed along the D1 direction as a pair of electrodes 36. As illustrated in FIG. 10B, the selection unit 50 may select two electrodes 36 disposed along the D3 direction as a pair of electrodes 36.

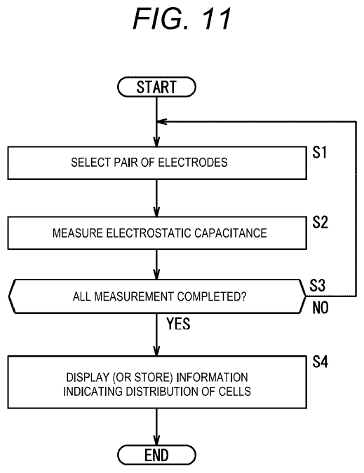

FIG. 11 is a flowchart illustrating the operation of the distribution measurement device 10 as illustrated in FIG. 1.

In step S1, the selection unit 50 may select a pair of electrodes 36 from the plurality of electrodes 36. For example, when the assembled electrode 38 is formed in advance, the selection unit 50 may select the assembled electrode 38 for which measurement of the electrostatic capacitance is not completed.

In step S2, the measurement unit 52 may execute switching control of the power supply unit 46 so that power is supplied to the pair of electrodes 36 selected by the selection unit 50. The power supply unit 46 may perform switching according to a switching signal output from the measurement unit 52 and may apply a voltage between the pair of electrodes 36. The measurement unit 52 may measure electrostatic capacitance between the selected pair of electrodes 36.

In step S3, the determination unit 54 may determine whether the measurement of all the electrostatic capacitance is completed. If the measurement of all the electrostatic capacitance is completed (step S3: YES), the processing may proceed to step S4. Meanwhile, if the measurement of some of the electrostatic capacitance is not completed (step S3: NO), the processing may return to step S1.

When the processing proceeds from step S3 to step S4, the measurement unit 52 may convert the electrostatic capacitance into a cell concentration using a calibration curve that indicates a relationship between the electrostatic capacitance and the cell concentration. The storage unit 44 may store the calibration curve in advance. The display control unit 56 may execute display control for displaying information indicating a distribution of cells in the longitudinal direction (D1 direction) of the bioreactor 12. The display unit 48 may display information indicating the distribution of cells according to the display control. For example, the display unit 48 may display a graph or a numerical value as the information indicating the distribution of cells. Meanwhile, the storage control unit 58 may store, in the storage unit 44, information indicating the distribution of cells in the longitudinal direction (D1 direction) of the bioreactor 12.

The plurality of pairs of electrodes 36 may form the plurality of cell concentration measurement ranges 62 along the D1 direction in the bioreactor 12 such that the distribution of cells in the longitudinal direction of the bioreactor 12 may be measured. The distribution of cells in the longitudinal direction of the bioreactor 12 may contribute to optimization of cell culture.

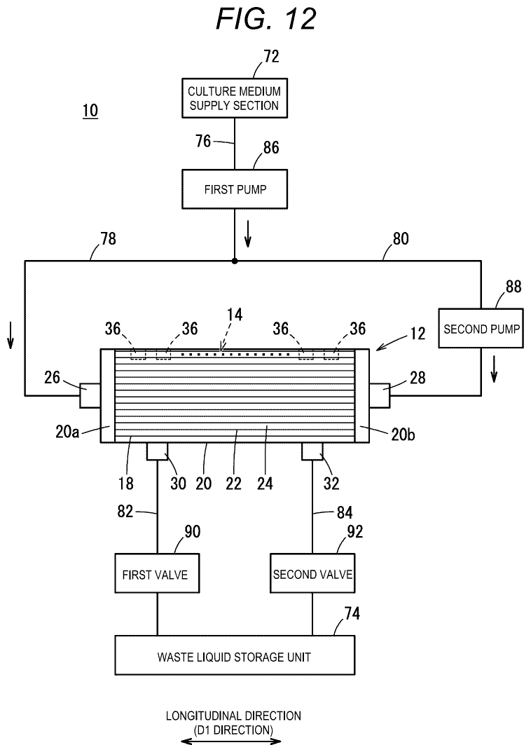

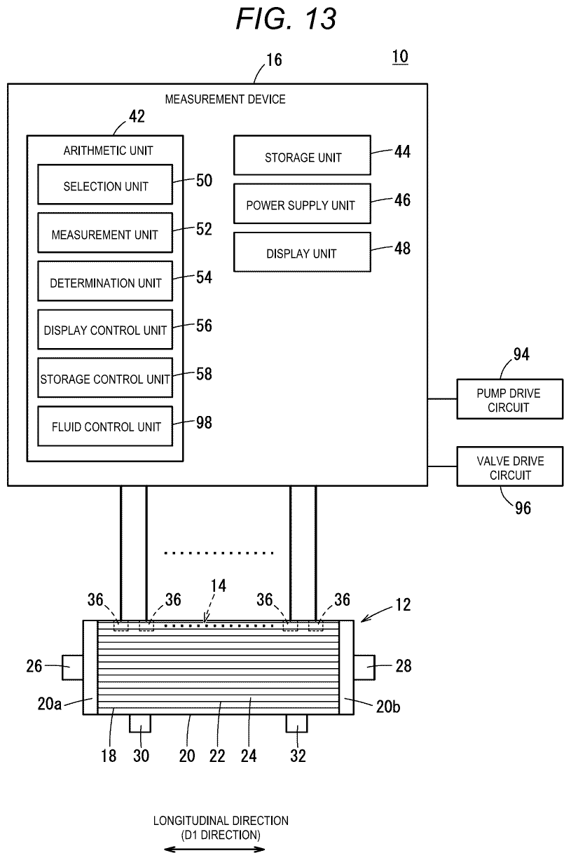

FIG. 12 is a fluid circuit diagram of another example distribution measurement device 10. FIG. 13 is a block diagram of the distribution measurement device 10 as illustrated in FIG. 12. The distribution measurement device 10 as illustrated in FIG. 12 can measure a distribution of cells in a longitudinal direction of the bioreactor 12. Further, when the cells to be cultured are floating cells, the distribution measurement device 10 as illustrated in FIG. 12 may change the distribution of the cells in the longitudinal direction of the bioreactor 12.

The distribution measurement device 10 as illustrated in FIG. 12 may be incorporated in a cell culture device that cultures cells. In such instances, some components of the cell culture device may be used as components of the distribution measurement device 10 as illustrated in FIG. 12. In the following description of the distribution measurement device 10 as illustrated in FIG. 12, components having the same configuration and functions as those of the distribution measurement device 10 as illustrated in FIG. 1 are denoted by the same reference signs and specific description thereof is omitted.

As illustrated in FIG. 12, the distribution measurement device 10 may include a culture medium supply section 72 and a waste liquid storage unit 74. The culture medium supply section 72 may include a medical bag filled with a culture medium. The waste liquid storage unit 74 may include a medical bag capable of collecting waste liquid discharged from the bioreactor 12.

The distribution measurement device 10 as illustrated in FIG. 12 may include a supply flow path 76, a first branch flow path 78, a second branch flow path 80, a first waste liquid flow path 82, and a second waste liquid flow path 84. Each of the flow paths may include a tube through which liquid flows. The supply flow path 76 may be connected to the culture medium supply section 72, the first branch flow path 78, and the second branch flow path 80. The first branch flow path 78 may be connected to the supply flow path 76 and the first port 26 of the bioreactor 12. The second branch flow path 80 may be connected to the supply flow path 76 and the second port 28 of the bioreactor 12. The first waste liquid flow path 82 may be connected to the third port 30 of the bioreactor 12 and the waste liquid storage unit 74. The second waste liquid flow path 84 may be connected to the fourth port 32 of the bioreactor 12 and the waste liquid storage unit 74.

The distribution measurement device 10 as illustrated in FIG. 12 may include a first pump 86 and a second pump 88. The first pump 86 may be disposed in the supply flow path 76. The second pump 88 may be disposed in the second branch flow path 80. The first pump 86 may apply a flow force to a culture medium in the supply flow path 76 in a direction toward the first branch flow path 78 and the second branch flow path 80. The second pump 88 may apply a flow force to a culture medium in the second branch flow path 80 in a direction toward the second port 28 or a direction toward the first port 26.

The distribution measurement device 10 as illustrated in FIG. 12 may include a first valve 90 and a second valve 92. The first valve 90 is disposed in the first waste liquid flow path 82. The second valve 92 may be disposed in the second waste liquid flow path 84. The first valve 90 and the second valve 92 may each include a clamp capable of opening and closing the flow path (tube).

As illustrated in FIG. 13, the arithmetic unit 42 may include a fluid control unit 98 (control unit). The fluid control unit 98 may be implemented by the arithmetic unit 42 executing a program stored in the storage unit 44.

The fluid control unit 98 may output various operation signals and controls an operation of the first pump 86, an operation of the second pump 88, an operation of the first valve 90, and an operation of the second valve 92. The fluid control unit 98 may control at least one of an inflow rate of a culture medium into the bioreactor 12 and an outflow rate of the culture medium from the bioreactor 12.

The distribution measurement device 10 as illustrated in FIG. 12 may include a pump drive circuit 94 and a valve drive circuit 96. The pump drive circuit 94 may supply power to the first pump 86 and the second pump 88 according to an operation signal output from the measurement device 16. The valve drive circuit 96 may supply power to the first valve 90 and the second valve 92 according to an operation signal output from the measurement device 16.

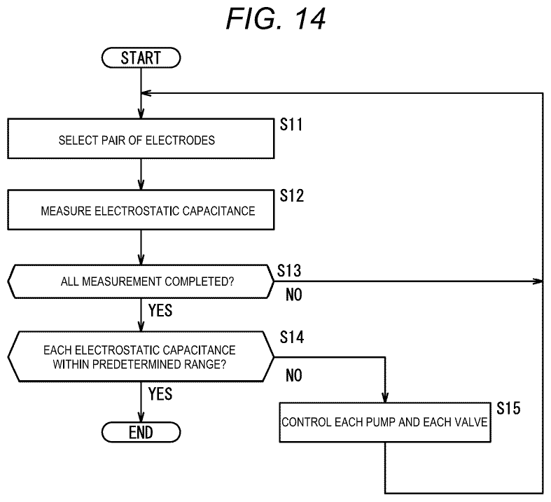

FIG. 14 is a flowchart illustrating the operation of the distribution measurement device 10 as illustrated in FIG. 12. A series of processes illustrated in FIG. 14 may be in a cell culture processing. The cell culture processing may include a cell agitation step of agitating cells inside the bioreactor 12 and/or a cell packing step of packing cells remaining in the flow path into the bioreactor 12 and/or a cell culture step of culturing the cells. The agitating, packing, and culturing steps of the cell culture processing may be repeatedly performed.

The series of processes illustrated in FIG. 14 may be performed in or during the cell packing step of the cell culture processing. As indicated by arrows in FIG. 12, in the cell packing step, the culture medium may be supplied from the culture medium supply section 72 to the bioreactor 12 via the supply flow path 76 and the first branch flow path 78. At the same time, in the cell packing step, the culture medium may be supplied from the culture medium supply section 72 to the bioreactor 12 via the supply flow path 76 and the second branch flow path 80. Accordingly, the cells remaining in the first branch flow path 78 and the second branch flow path 80 may be packed into the bioreactor 12.

Step S11 to step S13 illustrated in FIG. 14 are the same as step S1 to step S3 illustrated in FIG. 11.

In step S13, if the measurement of all the electrostatic capacitance is completed (step S13: YES), the processing may proceed to step S14. In step S14, the determination unit 54 may determine whether each measured electrostatic capacitance is within a predetermined range.

The predetermined range may be set based on the cell concentration in a state where the cells are uniformly diffused inside the bioreactor 12. A cell concentration in a state where cells are uniformly diffused may be referred to as a reference value. For example, the predetermined range may be set to a range between an upper limit value larger than the reference value by a predetermined value and a lower limit value smaller than the reference value by a predetermined value. The storage unit 44 may store the predetermined range in advance. If each electrostatic capacitance is within the predetermined range (step S14: YES), the series of processes illustrated in FIG. 14 may end. In such instances, the cell concentration at each position along the longitudinal direction of the bioreactor 12 may be substantially uniform. Meanwhile, if at least one electrostatic capacitance is out of the predetermined range (step S14: NO), the processing may proceed to step S15. In such instances, the cell concentration at each position along the longitudinal direction of the bioreactor 12 may be non-uniform.

When the processing proceeds from step S14 to step S15, the fluid control unit 98 may control each pump and each valve such that the cell concentration at positions along the longitudinal direction of the bioreactor 12 becomes uniform.

For example, a cell concentration at a position close to the first port 26 may be higher than a cell concentration at a position close to the second port 28. In such instances, the fluid control unit 98 may control the first pump 86 and the second pump 88 such that an inflow rate of the culture medium into the first port 26 is higher than an inflow rate of the culture medium into the second port 28. In such instances, a flow force from the first port 26 toward the second port 28 is larger than a flow force from the second port 28 toward the first port 26 inside the bioreactor 12. Further, the fluid control unit 98 may close the first valve 90 and may open the second valve 92. Then, inside the bioreactor 12, some of the cells diffusing near the first port 26 may move toward the second port 28. As a result, a bias of the cell concentration inside the bioreactor 12 may be corrected. The excess culture medium inside the bioreactor 12 may be discharged to the waste liquid storage unit 74 via the second waste liquid flow path 84.

Meanwhile, a cell concentration at a position close to the second port 28 may be higher than a cell concentration at a position close to the first port 26. In such instances, the fluid control unit 98 may control the first pump 86 and the second pump 88 such that an inflow rate of the culture medium into the second port 28 is higher than an inflow rate of the culture medium into the first port 26. In such instances, a flow force from the second port 28 toward the first port 26 is larger than a flow force from the first port 26 toward the second port 28 inside the bioreactor 12. Further, the fluid control unit 98 may close the second valve 92 and may open the first valve 90. Then, inside the bioreactor 12, some of the cells diffusing near the second port 28 may move toward the first port 26. As a result, a bias of the cell concentration inside the bioreactor 12 may be corrected. The excess culture medium inside the bioreactor 12 may be discharged to the waste liquid storage unit 74 via the first waste liquid flow path 82.

When a predetermined condition is satisfied after the processing of step S15 is started, the processing of step S11 and subsequent steps may be executed again. For example, when a supply amount of the culture medium in step S15 exceeds a predetermined amount, or when an execution time in step S15 exceeds a predetermined time, the processing of step S11 and subsequent steps may be executed again.

The fluid control unit 98 may control the pump and the valve based on the distribution of cells such that the distribution of cells in the longitudinal direction of the bioreactor 12 becomes uniform. When the distribution of the cells in the longitudinal direction of the bioreactor 12 is uniform, nutrition may be uniformly distributed to the cells inside the bioreactor 12. The uniformed distribution of the cells in the longitudinal direction of the bioreactor 12 may contribute to optimization of cell culture.

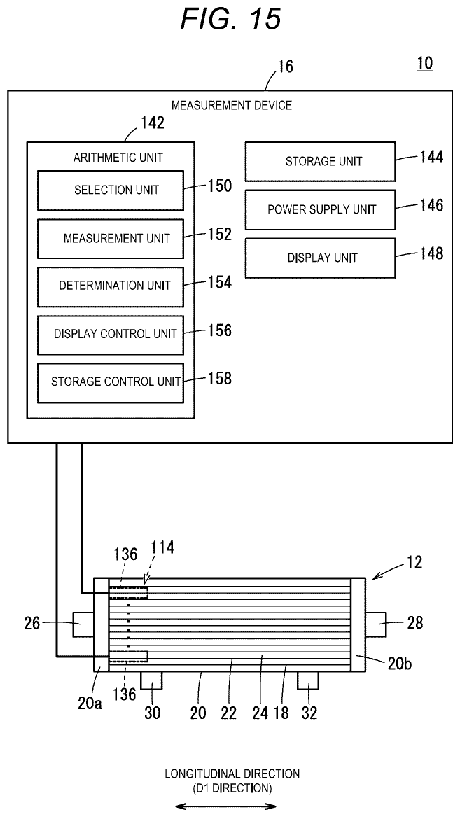

FIG. 15 is a block diagram of another example distribution measurement device 10. The distribution measurement device 10 as illustrated in FIG. 15 may measure a distribution of cells in a direction intersecting a longitudinal direction of the bioreactor 12. The distribution measurement device 10 as illustrated in FIG. 15 includes the bioreactor 12, an electrode group 114, and the measurement device 16.

In the following discussion of the distribution measurement device 10 as illustrated in FIG. 15, components having the same configuration and function as those of the distributed measurement device 10 as illustrated in FIG. 1 and/or the distributed measurement device 10 as illustrated in FIG. 12 are denoted by the same reference signs and specific description thereof is omitted.

The electrode group 114 may include a plurality of (three or more) electrodes 136. Each of the electrode 136 may be disposed inside the bioreactor 12. A longitudinal direction of the electrode 136 may be along the longitudinal direction of the bioreactor 12. That is, the electrode 136 may extend along the longitudinal direction of the bioreactor 12.

The measurement device 16 may measure electrostatic capacitance and/or dielectric constant between a pair of electrodes 136. The electrostatic capacitance between the pair of electrodes 136 is a physical quantity proportional to a cell concentration (i.e., the number of cells) between the pair of electrodes 136. In at least one example embodiment, the measurement device 16 may bean impedance analyzer or an LCR meter. The measurement device 16 may include, for example, an arithmetic unit 142, a storage unit 144, a power supply unit 146, and a display unit 148.

In at least one example embodiment, the arithmetic unit 142 may be implemented by a processor such as a central processing unit (CPU) or a graphics processing unit (GPU). That is, the arithmetic unit 142 may be implemented by a processing circuit.

The arithmetic unit 142 may include a selection unit 150, a measurement unit 152, a determination unit 154, a display control unit 156, and a storage control unit 158. Each of the selection unit 150, the measurement unit 152, the determination unit 154, the display control unit 156, and the storage control unit 158 may be implemented by the arithmetic unit 142 executing a program stored in the storage unit 144.

In at least one example embodiment, at least a part of the selection unit 150, the measurement unit 152, the determination unit 154, the display control unit 156, and the storage control unit 158 may be implemented by an integrated circuit such as an application specific integrated circuit (ASIC) or a field-programmable gate array (FPGA). In at least one example embodiment, at least a part of the selection unit 150, the measurement unit 152, the determination unit 154, the display control unit 156, and the storage control unit 158 may be implemented by an electronic circuit including a discrete device.

The selection unit 150 may select a pair of electrodes 136 from the plurality of electrodes 136. In at least one example embodiment, the selection unit 150 may sequentially select a pair of electrodes 136 from the plurality of electrodes 136. The measurement unit 152 may execute switching control of the electrode 136 based on a selection result of the selection unit 150. The measurement unit 152 may measure electrostatic capacitance between the pair of electrodes 136 selected by the selection unit 150. In at least one example embodiment, the measurement unit 152 may sequentially measure electrostatic capacitance between the pair of electrodes 136 selected by the selection unit 150. The determination unit 154 may perform various determinations. The display control unit 156 may execute display control. For example, the display control unit 156 may display (for example, on the display unit 148) information indicating a distribution of cells in the direction intersecting the longitudinal direction of the bioreactor 12 based on the electrostatic capacitance measured by the measurement unit 152. The storage control unit 158 may execute storage control. For example, the storage control unit 158 may store (for example, in the storage unit 144) information indicating the distribution of cells in the direction intersecting the longitudinal direction of the bioreactor 12 based on the electrostatic capacitance measured by the measurement unit 152.

The storage unit 144 may include a volatile memory (not illustrated) and a non-volatile memory (not illustrated). Examples of the volatile memory include a random access memory (RAM). The volatile memory may be used as a working memory of the processor and may temporarily store data and the like necessary for processing or calculation. Examples of the non-volatile memory include a read only memory (ROM) and a flash memory. The non-volatile memory may be used as a memory for storage and may store programs, tables, maps, and the like. In at least one example embodiment, at least a part of the storage unit 144 may be in the processor, the integrated circuit, or the like as described above.

The storage unit 144 may store information indicating the distribution of cells in the direction intersecting the longitudinal direction of the bioreactor 12 according to the storage control executed by the storage control unit 158 of the arithmetic unit 142. In at least one example embodiment, a part of the storage unit 144 may be provided outside the measurement device 16.

The power supply unit 146 may include a power supply circuit capable of applying a voltage (or current) between the pair of electrodes 136. The power supply circuit may include a plurality of switches. Each of the switches may be configured to switch connection and disconnection between a power supply (not illustrated) and the electrodes 136 according to a switching signal output from the arithmetic unit 142 (measurement unit 152).

The display unit 148 may include a drive circuit and a display. The display unit 148 may display information indicating the distribution of cells in the direction intersecting the longitudinal direction of the bioreactor 12 according to the display control executed by the arithmetic unit 142 (display control unit 156). For example, the display unit 148 may display the cell concentration in a numerical value. Alternatively, the display unit 148 may display the cell concentration as a graph.

The longitudinal direction of the bioreactor 12 is referred to as the D1 direction. Among radial directions of the bioreactor 12, two directions that are perpendicular to each other are referred to as the D2 direction and the D3 direction. The D2 direction and the D3 direction are perpendicular to the D1 direction.

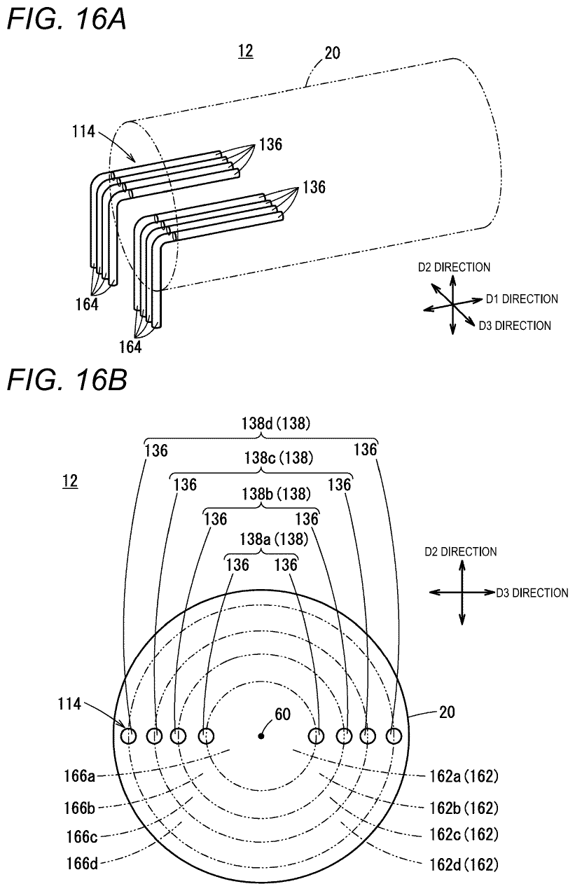

FIGS. 16A and 16B are schematic diagrams illustrating an example arrangement of the plurality of electrodes 136 of the bioreactor 12 as illustrated in FIG. 15. As illustrated in FIG. 16A, each of the electrodes 136 may extend along the D1 direction. The electrodes 136 may be disposed at the same position in the D1 direction. The electrode 136 may be connected to the power supply unit 146 as illustrated in FIG. 15 via a lead wire 164. As illustrated in FIG. 16B, a distance from each of two electrodes 136 of the plurality of electrodes 136 to the axis 60 of the housing 20 (bioreactor 12) may be equal. The two electrodes 136 may be spaced apart from each other to sandwich the axis 60 of the housing 20. That is, these two electrodes 136 and the axis 60 of the housing 20 may be disposed along the radial direction (for example, D3 direction) of the housing 20 (bioreactor 12). When the electrostatic capacitance is measured, these two electrodes 136 may be simultaneously selected. One of the two electrodes 136 may be a positive electrode and the other may be a negative electrode. That is, when the electrostatic capacitance is measured, these two electrodes 136 may form a pair. In this way, two electrodes 136 that form a pair in which only one electrode 136 may be correlated with one electrode 136 in advance are referred to as an “assembled electrode 138”. A plurality of assembled electrodes 138 may have different distances between the electrodes 136 (inter-electrode distances) are provided inside the bioreactor 12.

As illustrated in FIGS. 16A and 16B, the plurality of assembled electrodes 138 may be disposed along the same direction (D3 direction). That is, the plurality of electrodes 136 may be disposed along the direction (D3 direction) perpendicular to the longitudinal direction (D1 direction) of the bioreactor 12. In other example embodiments, the plurality of electrodes 136 may be disposed along a direction intersecting the longitudinal direction (D1 direction) of the bioreactor 12 as illustrated in FIG. 15 other than the D3 direction.

When the power supply unit 146 as illustrated in FIG. 15 applies a voltage (or a current) to the assembled electrode 138 (the pair of electrodes 136), the assembled electrode 138 may store a charge corresponding to a cell concentration between the electrodes 136. The cell concentration may be substantially constant in a certain range centered on the assembled electrode 138. The assembled electrode 138 may function as a sensor member for measuring the cell concentration in a certain range. This certain range may be referred to as a measurement range 162. As illustrated in FIG. 16B, the measurement range 162 may have a substantially circular shape centered on the axis 60 of the housing 20 in the side view.

As illustrated in FIGS. 16A and 16B, four assembled electrodes 138 may be provided inside the bioreactor 12. Although four assembled electrodes 138 are illustrated, it should be appreciated that the number of assembled electrodes 138 may be two or more. Each of the electrodes 136 may be disposed on any one of four concentric circles centered on the axis 60 of the housing 20. The electrode 136 in a first assembled electrode 138a may be disposed on a circle having the smallest diameter. That is, an inter-electrode distance of the first assembled electrode 138a may be the smallest among inter-electrode distances of the four assembled electrodes 138. The electrode 136 in a second assembled electrode 138b may be disposed on a circle having the second smallest diameter. That is, an inter-electrode distance of the second assembled electrode 138b may be the second smallest among the inter-electrode distances of the four assembled electrodes 138. The electrode 136 in a third assembled electrode 138c may be disposed on a circle having the third smallest diameter. That is, an inter-electrode distance of the third assembled electrode 138c may be the third smallest among the inter-electrode distances of the four assembled electrodes 138. The electrode 136 in a fourth assembled electrode 138d may be disposed on a circle having the largest diameter. That is, an inter-electrode distance of the fourth assembled electrode 138d may be the largest among the inter-electrode distances of the four assembled electrodes 138. The electrode 136 in the fourth assembled electrode 138d may be in contact with the inner peripheral surface of the housing 20.

Inside the bioreactor 12, four measurement ranges 162 may be formed by the four assembled electrodes 138. The measurement range 162 (first measurement range 162a) of the first assembled electrode 138a may be the smallest. The measurement range 162 (second measurement range 162b) of the second assembled electrode 138b may be one size larger than the first measurement range 162a. The measurement range 162 (third measurement range 162c) of the third assembled electrode 138c may be one size larger than the second measurement range 162b. The measurement range 162 (fourth measurement range 162d) of the fourth assembled electrode 138d may be one size larger than the third measurement range 162c.

The first measurement range 162a may also referred to as a first distribution range 166a. A range of the second measurement range 162b other than the first measurement range 162a may be referred to as a second distribution range 166b. A range of the third measurement range 162c other than the second measurement range 162b may be referred to as a third distribution range 166c. A range of the fourth measurement range 162d other than the third measurement range 162c may be referred to as a fourth distribution range 166d. The cell concentration in these four distribution regions may indicate a distribution of cells in a cross section perpendicular to the longitudinal direction of the bioreactor 12.

The number of cells in the measurement range 162 may be acquired based on the electrostatic capacitance in the measurement range 162. The number of cells in the first distribution range 166a may be acquired based on electrostatic capacitance in the first measurement range 162a. The number of cells in the second distribution range 166b may be acquired by subtracting the number of cells in the first measurement range 162a from the number of cells in the second measurement range 162b. The number of cells in the third distribution range 166c may be acquired by subtracting the number of cells in the second measurement range 162b from the number of cells in the third measurement range 162c. The number of cells in the fourth distribution range 166d may be acquired by subtracting the number of cells in the third measurement range 162c from the number of cells in the fourth measurement range 162d.

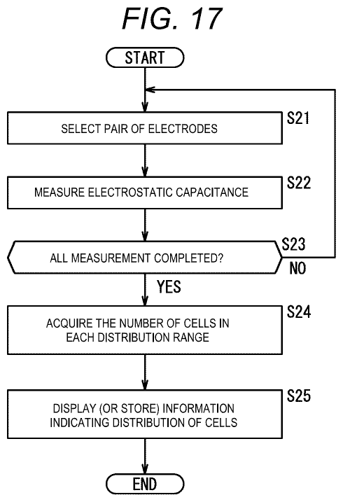

FIG. 17 is a flowchart illustrating the operation of the distribution measurement device 10 as illustrated in FIG. 15.

In step S21, the selection unit 150 may select a pair of electrodes 136 from the plurality of electrodes 136. For example, when the assembled electrode 138 is formed in advance, the selection unit 150 may select the assembled electrode 138 for which measurement of the electrostatic capacitance is not completed.

In step S22, the measurement unit 152 may execute switching control of the power supply unit 146 so that power is supplied to the pair of electrodes 136 selected by the selection unit 150. The power supply unit 146 may perform switching according to a switching signal output from the measurement unit 152 and may apply a voltage between the pair of electrodes 136. The measurement unit 152 may measure electrostatic capacitance between the selected pair of electrodes 136.

In step S23, the determination unit 154 may determine whether the measurement of all the electrostatic capacitance is completed. If the measurement of all the electrostatic capacitance is completed (step S23: YES), the processing may proceed to step S24. Meanwhile, if the measurement of some of the electrostatic capacitance is not completed (step S23: NO), the processing may return to step S21.

When the processing proceeds from step S23 to step S24, the measurement unit 152 may convert the electrostatic capacitance into a cell concentration using a calibration curve that indicates a relationship between the electrostatic capacitance and the cell concentration. The storage unit 144 may store the calibration curve in advance. In a case of the arrangement of the electrodes 136 illustrated in FIGS. 16A and 16B, the measurement unit 152 may acquire the number of cells in each of the first distribution range 166a to the fourth distribution range 166d based on the cell concentration in each of the first measurement range 162a to the fourth measurement range 162d.

In step S25, the display control unit 156 may execute display control for displaying information indicating a distribution of cells in a direction intersecting the longitudinal direction (D1 direction) of the bioreactor 12. The display unit 148 may display information indicating the distribution of cells according to the display control. For example, the display unit 148 may display a graph or a numerical value as the information indicating the distribution of cells. Meanwhile, the storage control unit 158 may store, in the storage unit 144, information indicating the distribution of cells in the direction intersecting the longitudinal direction (D1 direction) of the bioreactor 12.

The longitudinal direction of the electrode 136 for measuring the electrostatic capacitance may be along the longitudinal direction of the bioreactor 12. By sequentially switching a combination of the electrodes 136, the distribution of cells in the direction intersecting the longitudinal direction of the bioreactor 12 may be measured. The distribution of cells in the direction intersecting the longitudinal direction of the bioreactor 12 may contribute to optimization of cell culture.

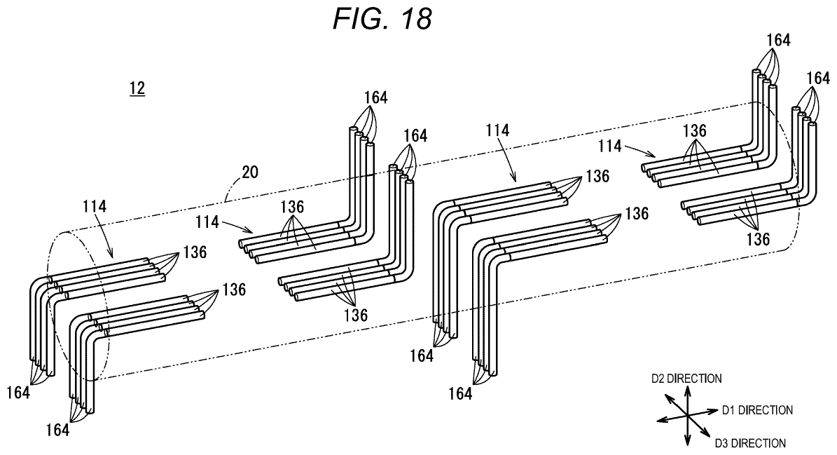

FIG. 18 is a schematic diagram illustrating an example arrangement of the plurality of electrodes 136 in the bioreactor 12 as illustrated in FIG. 15. As illustrated in FIG. 18, a plurality of electrodes 136 (a plurality of assembled electrodes 138) may be disposed at a plurality of positions in the longitudinal direction (D1 direction) of the bioreactor 12.

A distribution of cells in the longitudinal direction (D1 direction) of the bioreactor 12 may be further measured.

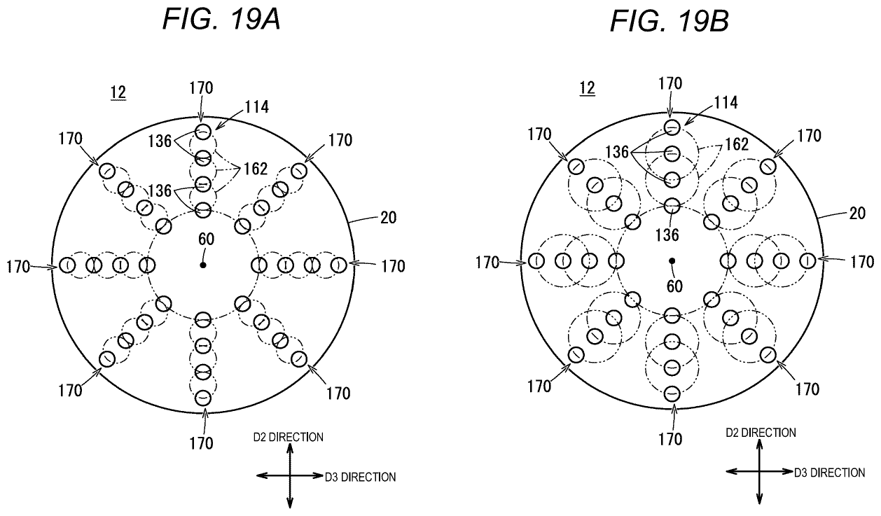

FIGS. 19A and 19B are schematic diagrams illustrating another example arrangement of a plurality of electrodes 136 in the bioreactor 12 as illustrated in FIG. 15. AS illustrated in FIGS. 19A and 19B, the electrode group 114 may include eight small electrode groups 170. Each of the eight small electrode groups 170 may include a plurality of electrodes 136. In FIGS. 19A and 19B, the small electrode group 170 may include four electrodes 136. A longitudinal direction of the electrode 136 may be along the longitudinal direction of the bioreactor 12. That is, the electrode 136 may extend along the longitudinal direction of the bioreactor 12. The electrode 136 may be connected to the power supply unit 146 as illustrated in FIG. 15. The electrodes 136 may be disposed at the same position in the D1 direction.

In the small electrode group 170, the plurality of electrodes 136 and the axis 60 of the housing 20 may be disposed along the radial direction of the housing 20. A disposition direction of the small electrode group 170 may be shifted by 45 degrees from a disposition direction of the adjacent small electrode groups 170 about the axis 60 of the housing 20.

The selection unit 150 as illustrated in FIG. 15 may randomly select two electrodes 136 to form a pair of electrodes 136. For example, as illustrated in FIG. 19A, the selection unit 150 may sequentially select electrodes 136 adjacent to each other from one small electrode group 170 as a pair of electrodes 136. As illustrated in FIG. 19B, the selection unit 150 may sequentially select two electrodes 136 sandwiching one or a plurality of electrodes 136 from one small electrode group 170 as a pair of electrodes 136. As illustrated in FIG. 19B, the selection unit 150 may sequentially select a pair of electrodes 136 from one small electrode group 170 such that a plurality of measurement ranges 162 overlap each other. As in the third embodiment, the selection unit 150 may select two electrodes 136 that are spaced apart from each other and sandwich the axis 60 therebetween.

The pair of electrodes 136 may be formed in various combinations. Therefore, the distribution of cells in the direction intersecting the longitudinal direction of the bioreactor 12 may be measured in more detail than in the third embodiment.

1. A distribution measurement device comprising:
a plurality of electrodes disposed in a bioreactor;
a selection unit configured to select a pair of the electrodes from the plurality of electrodes; and
a measurement unit configured to measure a physical quantity between the pair of electrodes.

2. The distribution measurement device of claim 1, wherein the plurality of electrodes are disposed along a longitudinal direction of the bioreactor.

3. The distribution measurement device of claim 2, wherein the plurality of electrodes are disposed at equal intervals along the longitudinal direction of the bioreactor.

4. The distribution measurement device of claim 2, wherein each consecutive pair of electrodes of the plurality of electrodes are disposed apart from each other in the longitudinal direction along the bioreactor.

5. The distribution measurement device of claim 2, wherein a first electrode of a first pair of electrodes of the plurality of electrodes is spaced from a second electrode of the first pair of electrode of the plurality of electrode by at least one of the electrodes of a second pair of electrodes of the plurality of electrodes.

6. The distribution measurement device of claim 2, wherein the bioreactor includes a plurality of convex portions along the longitudinal direction, each of the convex portions of the plurality of convex portions extending away from an outer peripheral surface of a housing of the bioreactor, each of the plurality of convex portions being associated with at least one electrode of the plurality of electrodes.

7. The distribution measurement device of claim 6, wherein the plurality of convex portions includes a first group of convex portions extending away from a first outer peripheral surface of the housing of the bioreactor and a second group of convex portions extending away from a second outer peripheral surface of the housing of the bioreactor.

8. The distribution measurement device of claim 2, wherein a first electrode of the pair of electrodes is disposed on a first side of a major axis of the bioreactor and a second electrode of the pair of electrodes is disposed on second side of the major axis of the bioreactor.

9. The distribution measurement device of claim 2, wherein the bioreactor includes a hollow fiber,
a longitudinal direction of the hollow fiber is along the longitudinal direction of the bioreactor.

10. The distribution measurement device of claim 2, wherein a longitudinal direction of each of the electrodes of the plurality of electrodes intersects the longitudinal direction of the bioreactor.

11. The distribution measurement device of claim 10, further comprising:
a display control unit that is configured to display information indicating a distribution of cells in the longitudinal direction of the bioreactor based on the physical quantity as measured by the measurement unit.

12. The distribution measurement device of claim 10, further comprising:
a storage control unit that is configured to store information that indicates a distribution of cells in the longitudinal direction of the bioreactor based on the physical quantity as measured by the measurement unit.

13. The distribution measurement device of claim 1, wherein a longitudinal direction of each of the electrodes of the plurality of electrodes is along a longitudinal direction of the bioreactor.

14. The distribution measurement device according to claim 13, further comprising:
a display control unit that is configured to display information indicating a distribution of cells in a direction intersecting the longitudinal direction of the bioreactor based on the physical quantity sequentially measured by the measurement unit.

15. The distribution measurement device according to claim 13, further comprising:
a storage control unit that is configured to store information indicating a distribution of cells in a direction intersecting the longitudinal direction of the bioreactor based on the physical quantity sequentially measured by the measurement unit.

16. The distribution measurement device of claim 2, further comprising:
a control unit that is configured to control at least one of an inflow rate of a culture medium into the bioreactor and an outflow rate of the culture medium from the bioreactor such that a distribution of cells in the longitudinal direction of the bioreactor becomes uniform.

17. The distribution measurement device of claim 1, wherein the bioreactor includes a plurality of supply and discharge ports, a first supply and discharge port of the plurality of supply and discharge ports being provided at one end of the bioreactor and a second supply and discharge port of the plurality of supply and discharge ports being provided at the other end of the bioreactor.

18. The distribution measurement device of claim 1, wherein a first electrode of the pair of electrodes is a positive electrode and a second electrode of the pair of electrodes is a negative electrode.

19. The distribution measurement device of claim 1, wherein the physical quantity is electrostatic capacitance.

20. A distribution measurement device comprising:
a plurality of electrodes disposed along a longitudinal direction of a bioreactor, the bioreactor including a hollow fiber and a longitudinal direction of the bioreactor defined by a longitudinal direction of the hollow fiber;
a selection unit configured to select a pair of the electrodes from the plurality of electrodes, a first electrode of the pair of electrodes being a positive electrode and a second electrode of the pair of electrodes being a negative electrode; and
a measurement unit that is configured to measure an electrostatic capacitance between the pair of electrodes.
